(19) United States
(12) Patent Application Publication
(10) Pub. No.: US 2024/0382534 A1
(43) Pub. Date: Nov. 21, 2024

(54) MITOCHONDRIAL AUGMENTATION THERAPY FOR MYELODYSPLASTIC SYNDROME

(71) Applicant: Minovia Therapeutics Ltd., Tirat Hacarmel (IL)

(72) Inventors: Natalie Yivgi-Ohana, Haifa (IL); Noa Sher, Haifa (IL)

(21) Appl. No.: 18/714,544

(22) PCT Filed: Dec. 1, 2022
(86) PCT No.: PCT/IL2022/051280

§ 371 Date: May 29, 2024

Related U.S. Application Data
(60) Provisional application No. 63/289,069, filed on Dec. 13, 2021.

(51) Int. Cl. A61K 35/545; A61K 35/12; A61K 35/28; A61K 35/51; A61K 45/06; A61P 7/00

(52) U.S. Cl. CPC A61K 35/545; A61K 35/28; A61K 35/51; A61K 45/06; A61P 7/00; A61K 2035/124

(57) ABSTRACT

The present disclosure provides cells enriched with mitochondria to treat myelodysplastic syndrome (MDS). diseases and disorders. The disclosure provides pharmaceutical compositions of mitochondrially-enriched stem cells and/or progenitor cells. methods of treatment of myelodysplastic syndrome. and methods to alleviate symptoms of MDS and/or prevent the progression of MDS using mitochondrially-enriched stem cells and/or progenitor cells.

### CROSS-REFERENCE TO RELATED APPLICATIONS

This application claims benefit of priority under 35 U.S.C. § 119 (e) of U.S. Provisional Application No. 63/289,069, filed Dec. 13, 2021. The disclosure of the prior application is considered part of and is herein incorporated by reference in the disclosure of this application in its entirety.

### BACKGROUND OF THE INVENTION

### Field of the Invention

The present invention relates generally to cells enriched with mitochondria and more specifically to mitochondria-enriched stem cell and/or progenitor cell compositions and methods of use thereof for the treatment of myelodysplastic syndrome.

### Background Information

The mitochondrion is a membrane bound organelle found in most eukaryotic cells, ranging from 0.5 to 1.0 μm in diameter. Mitochondria are found in nearly all eukaryotic cells and vary in number and location depending on the cell type. Mitochondria contain their own DNA (mtDNA) and their own machinery for synthesizing RNA and proteins. The mtDNA contains only 37 genes, thus most of the gene products in the mammalian body are encoded by nuclear DNA.

Mitochondria perform numerous essential tasks in the eukaryotic cell such as pyruvate oxidation, the Krebs cycle and metabolism of amino acids, fatty acids and steroids. However, the primary function of mitochondria is the generation of energy as adenosine triphosphate (ATP) by means of the electron-transport chain and the oxidative-phosphorylation system (the “respiratory chain”). Additional processes in which mitochondria are involved include heat production, storage of calcium ions, calcium signaling, programmed cell death (apoptosis) and cellular proliferation.

The ATP concentration inside the cell is typically 1-10 mM ATP and can be produced by redox reactions using simple and complex sugars (carbohydrates) or lipids as an energy source. For complex fuels to be synthesized into ATP, they first need to be broken down into smaller, simpler molecules. Complex carbohydrates are hydrolyzed into simple sugars, such as glucose and fructose. Fats (triglycerides) are metabolized to give fatty acids and glycerol.

The overall process of oxidizing glucose to carbon dioxide is known as cellular respiration and can produce about 30 molecules of ATP from a single molecule of glucose. ATP can be produced by a number of distinct cellular processes. The three main pathways used to generate energy in eukaryotic organisms are glycolysis and the citric acid cycle/oxidative phosphorylation, both components of cellular respiration, and beta-oxidation.

The majority of this ATP production by non-photosynthetic eukaryotes takes place in the mitochondria, which can make up nearly 25% of the total volume of a typical cell.

Myelodysplastic syndrome (MDS) is defined by ineffective hematopoiesis resulting in blood cytopenia, and clonal instability with a risk of clonal evolution to acute myeloid leukemia (AML). Patients with MDS collectively have a high symptom burden and are also at risk of death from complications of cytopenia and AML. The goals of therapy for patients with MDS are to reduce disease-associated symptoms and the risk of disease progression and death, thereby improving both quality and length of life. Unfortunately, therapeutic options remain limited to supportive care with transfusions, growth factors, and a limited number of approved drugs that are currently available.

The main disadvantages of the currently available treatments are the limited response rate, the short durability of effect, the limited treatment options for neutropenia and thrombocytopenia and the fact that except for hematopoietic cell transplant (HCT), treatments are not curative. The majority of patients will eventually relapse, and current treatments do not prevent progression. Therefore, there is a critical need for a new treatment as a stand-alone or combination treatment especially with a new mechanism of action.

Mitochondrial augmentation therapy (MAT) is a cell therapy comprising autologous stem and/or progenitor cells (HSPCs) enriched with healthy mitochondria. MAT offers a therapeutic modality for restoring the function of the hematopoietic linage by having the mitochondrially enriched cells colonize the bone marrow to establish new hematopoietic colonies of stem cells and/or progenitor cells with healthy mitochondria. MAT can be used as a stand-alone therapy or in combination with current standard of care to reduce overall disease burden and healthcare utilization and to improve quality of life. Therefore, MAT addresses a highly unmet medical need for patients with myelodysplastic syndrome.

### SUMMARY OF THE INVENTION

The present invention is based on the seminal discovery that cells enriched with mitochondria are useful for treating myelodysplastic syndrome (MDS), diseases and disorders. The present invention provides pharmaceutical compositions of mitochondrially-enriched stem cells and/or progenitor cells, methods of treatment of myelodysplastic syndrome, and methods to alleviate symptoms of MDS and/or prevent the progression of MDS using mitochondrially-enriched stem cells and/or progenitor cells.

In one embodiment, the present invention provides a pharmaceutical composition including stem cells and/or progenitor cells enriched with exogenous mitochondria and a pharmaceutically acceptable carrier, wherein the stem cells and/or progenitor cells are obtained from a subject having a myelodysplastic syndrome (MDS) disease, disorder or symptom thereof, and wherein the exogenous mitochondria are obtained from a donor that does not have a MDS disease, disorder or symptom thereof or a mitochondrial disease.

In one aspect, the stem cells and/or progenitor cells enriched with exogenous mitochondria are produced by a method including contacting the stem cells and/or progenitor cells with the exogenous mitochondria under conditions allowing the exogenous mitochondria to enter the stem cells and/or progenitor cells. In some aspects, the conditions allowing the exogenous mitochondria to enter the stem cells and/or progenitor cells include incubating the stem cells and/or progenitor cells with the exogenous mitochondria for a time ranging from about 0.5 to 30 hours at a temperature ranging from about 4 to 37° C. In one aspect, the conditions allowing the exogenous mitochondria to enter the target cells include incubating the target cells with the exogenous mitochondria at a ratio of about 0.044-176 mU citrate synthase (CS) activity per 10⁶ cells. In some aspects, the conditions allowing the exogenous mitochondria to enter the target cells include incubating the target cells with the exogenous mitochondria at a concentration of about 1-50 mU citrate synthase (CS) activity per 10⁶ cells. In various aspects, the concentration of exogenous mitochondria is about 4.4, 17.6, or 35 mU citrate synthase (CS) activity per 10⁶ cells. In one embodiment, the conditions allowing the exogenous mitochondria to enter the target cells by incubating the target cells with the exogenous mitochondria at a ratio of about 1 up to 200 mitochondria particles per cell. In certain embodiments, the conditions allowing the exogenous mitochondria to enter the target cells comprise incubating the target cells with the exogenous mitochondria at a ratio of about 10 up to 50 mitochondria particles per cell.

In another aspect, the stem cells and/or progenitor cells enriched with exogenous mitochondria have: an increased content of at least one mitochondrial protein; an increased rate of oxygen (O₂) consumption; an increased activity level of citrate synthase, succinate, or tryptamine; an increase rate of adenosine triphosphate (ATP) production; an increased mitochondrial DNA content; increased colony forming units activity in liquid medium or solid medium; an increased rate of proliferation; an increased rate of differentiation; or any combination thereof, as compared to the stem cells and/or progenitor cells prior to mitochondrial enrichment. In one aspect, the exogenous mitochondria constitute at least 0.5% of the total mitochondria in the stem cells and/or progenitor cells enriched with exogenous mitochondria. In another aspect, the exogenous mitochondria are isolated, derived or partially purified from human placenta. In another aspect, the composition is frozen/thawed. In some aspects, the stem cells and/or progenitor cells are frozen/thawed prior to the enrichment. In other aspects, the exogenous mitochondria are frozen/thawed prior to the enrichment of the stem cells and/or progenitor cells. In one aspect, the stem cells and/or progenitor cells have undergone at least one freeze-thaw cycle after enrichment with the exogenous mitochondria. In another aspect, the stem cells and/or progenitor cells are selected from pluripotent stem cells, embryonic stem cells, induces pluripotent stem cells, hematopoietic stem cells, hematopoietic progenitor cells, common myeloid progenitor cells, common lymphoid progenitor cells, CD34 ⁺ cells, a subset of CD34 ⁺ cells, and any combination thereof. In various aspects, the stem cells and/or progenitor cells are CD34 ⁺ cells or a subset of CD34 ⁺ cells. In one aspect, the stem cells and/or progenitor cells are derived from whole blood, blood fractions, peripheral blood, umbilical blood, bone marrow or bone marrow cells mobilized to the blood. In one aspect, the exogenous mitochondria are isolated, derived or purified from placenta, placental cells grown in culture, or blood cells.

In another aspect, the MDS disease or disorder is selected from the group myelodysplastic syndrome with single-lineage dysplasia (MDS-SLD), myelodysplastic syndrome with multilineage dysplasia (MDS-MLD), myelodysplastic syndrome with ring sideroblasts (MDS-RS), myelodysplastic syndrome with isolated del(5q), myelodysplastic syndrome with excess blasts (MDS-EB), myelodysplastic syndrome, unclassifiable (MDS-U), and acute myeloid leukemia (AML). In one aspect, the MDS symptom is selected from the group consisting of shortness of breath, weakness, fatigue, paleness, anemia, thrombocytopenia, leukopenia, bruising, bleeding, petechiae, ineffective hematopoiesis, blood cytopenia, clonal instability, and any combination thereof. In another aspect, the subject is treated or has been treated with a MDS treatment. In some aspects, the MDS treatment is a hypomethylating agent, an erythropoiesis stimulating agent (ESA), granulocyte colony-stimulating factor (G-CSF), azacytidine, decitabine, an immunosuppressive therapy (IST), luspatercept, or a combination thereof.

In another embodiment, mitochondrial diseases are a group of disorders caused by dysfunctional mitochondria. Mitochondrial diseases may be caused by mutations in the mitochondrial DNA that affect mitochondrial function. Other causes of mitochondrial disease are mutations in genes of the nuclear DNA, whose gene products are imported into the Mitochondria (Mitochondrial proteins) as well as acquired mitochondrial conditions. Mitochondrial diseases take on unique characteristics both because of the way the diseases are often inherited and because mitochondria are so critical to cell function. The subclass of these diseases that have neuromuscular disease symptoms are often called a mitochondrial myopathy.

In another embodiment, the invention provides a method of reducing a myelodysplastic syndrome (MDS) disease or disorder-associated symptom in a subject including administering to the subject stem cells and/or progenitor cells enriched with exogenous mitochondria. In one aspect, the subject has one or more MDS disease-or disorder-associated symptoms. In various aspects, the one or more MDS disease-or disorder-associated symptoms are selected from the group consisting of shortness of breath, weakness, fatigue, paleness, anemia, thrombocytopenia, leukopenia, bruising, bleeding, petechiae, ineffective hematopoiesis, blood cytopenia, bone marrow dysplasia, lymphopenia clonal instability, and any combination thereof.

In an additional embodiment, the invention provides a method of preventing myelodysplastic syndrome (MDS) disease or disorder progression in a subject including administering to the subject stem cells and/or progenitor cells enriched with exogenous mitochondria.

In a further embodiment, the invention provides a method of treating a myelodysplastic syndrome (MDS) disease or disorder in a subject including administering to the subject stem cells and/or progenitor cells enriched with exogenous mitochondria.

In one aspect, administering stem cells and/or progenitor cells enriched with exogenous mitochondria prevents disease progression and/or improves survival.

In one embodiment, the invention provides a method of restoring hematopoietic lineage function or cell count in a subject in need thereof including administering to the subject stem cells and/or progenitor cells enriched with exogenous mitochondria. In certain embodiments, restoring hematopoietic function or cell count is characterized by an improved cell differentiation, an improvement of anemia, reduction in the number of blast cells, a reduction in the number of ring sideroblasts and/or a reduction for the need for a blood transfusion.

In one aspect, the improved cell differentiation comprises improved CD34 ⁺ cells erythroid differentiation.

In one aspect, the stem cells and/or progenitor cells are autologous. In one aspect, the stem cells and/or progenitor cells are allogeneic. In another aspect, the stem cells and/or progenitor cells enriched with exogenous mitochondria colonize the bone marrow to establish new hematopoietic colonies. In another aspect, the method further includes administering to the subject a MDS treatment selected from a hypomethylating agent, an erythropoiesis stimulating agent (ESA), an erythroid maturation agent, an immunosuppressive therapy (IST), a growth factor, an immunomodulatory drug, a nucleoside analog, a blood transfusion, a bone marrow transplant, or a combination thereof. In some aspects, the MDS treatment is selected from the group consisting of azacytidine, decitabine, cedazuridine, luspatercept, granulocyte colony-stimulating factor (G-CSF), lenalidomide, epoetin and darbepoetin, or a combination thereof. In one aspect, the exogenous mitochondria constitute at least 1% of the total mitochondria content in the mitochondrially-enriched stem cells and/or progenitor cells. In another aspect, administration of mitochondrially-enriched stem cells and/or progenitor cells is by intravenous, intraperitoneal, intraarterial or intramuscular administration or direct injection to the bone marrow. In one aspect, between at least 5×10⁵ to 5×10⁹ mitochondrially-enriched stem cells and/or progenitor cells are administered to the subject.

### BRIEF DESCRIPTION OF THE DRAWINGS

FIG. 1 is a schematic representation of the protocol for augmenting whole bone marrow as described herein.

FIG. 2 shows the effects of MAT augmentation on MDS cells differentiation and proliferation.

FIGS. 3A-3B show erythroid cell counts in low and intermediate-risk MDS cells, before and after MAT augmentation. FIG. 3A is a graph showing erythroid cell count in CD34 purified cells. FIG. 3B is a graph showing erythroid cell count in whole bone barrow cells.

### DETAILED DESCRIPTION OF THE INVENTION

The present invention is based on the seminal discovery that cells enriched with mitochondria are useful for treating myelodysplastic syndrome (MDS), diseases and disorders. The present invention provides pharmaceutical compositions of mitochondrially-enriched stem cells and/or progenitor cells, methods of treatment of myelodysplastic syndrome, and methods or alleviate symptoms of MDS and/or prevent the progression of MDS using mitochondrially-enriched stem cells and/or progenitor cells.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

As used herein, the term “about” in association with a numerical value is meant to include any additional numerical value reasonably close to the numerical value indicated. For example, and based on the context, the value can vary up or down by 5-10%. For example, for a value of about 100, means 90 to 110 (or any value between 90 and 110).

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

In one embodiment, the present invention provides a pharmaceutical composition including stem cells and/or progenitor cells enriched with exogenous mitochondria and a pharmaceutically acceptable carrier, wherein the stem cells and/or progenitor cells are obtained from a subject having a myelodysplastic syndrome (MDS) disease, disorder or symptom thereof, and wherein the exogenous mitochondria are obtained from a donor that does not have a MDS disease, disorder or symptom thereof.

As used herein, the term “pharmaceutical composition” refers to a formulation including an active ingredient, and optionally a pharmaceutically acceptable carrier, diluent or excipient. The term “active ingredient” can interchangeably refer to an “effective ingredient” and is meant to refer to any agent that is capable of inducing a sought-after effect upon administration. Examples of active ingredient include, but are not limited to, cells or biologic tissue, chemical compound, drug, therapeutic agent, small molecule, etc.

The pharmaceutical compositions described herein include stem cells and/or progenitor cells enriched with exogenous mitochondria. As used herein the term, “stem cells and/or progenitor cells enriched with exogenous mitochondria” can be used interchangeably with the terms “mitochondrially-enriched stem cells”, or “mitochondrially-enriched progenitor cell”, and refers to a population of stem cell contacted with exogenous mitochondria thereby some or all of the stem cells comprise exogenous mitochondria.

As used herein, the term “stem cells” generally refers to any mammalian stem cells. Stem cells are undifferentiated cells that can differentiate into other types of cells and can divide to produce more of the same type of stem cells. Stem cells can be either totipotent or pluripotent. “Stem cells” generally refers to all stem cells naturally found in a subject, and to all stem cells produced or derived ex vivo. A “progenitor cell”, like a stem cell, has a tendency to differentiate into a specific type of cell, but is already more specific than a stem cell and is pushed to differentiate into its “target” cell. The most important difference between stem cells and progenitor cells is that stem cells can replicate indefinitely, whereas progenitor cells can divide only a limited number of times. The term “human stem cells” as used herein further includes “progenitor cells” and “non-fully differentiated stem cells”.

By “pharmaceutically acceptable” it is meant the carrier, diluent or excipient must be compatible with the other ingredients of the formulation and not deleterious to the recipient thereof, nor to the activity of the active ingredient of the formulation. Pharmaceutically acceptable carriers, excipients or stabilizers are well known in the art, for example Remington's Pharmaceutical Sciences, 16th edition, Osol, A. Ed. (1980). Pharmaceutically acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine: monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG). Examples of carrier include, but are not limited to, liposome, nanoparticles, ointment, micelles, microsphere, microparticle, cream, emulsion, and gel. Examples of excipient include, but are not limited to, anti-adherents such as magnesium stearate, binders such as saccharides and their derivatives (sucrose, lactose, starches, cellulose, sugar alcohols and the like) protein like gelatin and synthetic polymers, lubricants such as talc and silica, and preservatives such as antioxidants, vitamin A, vitamin E, vitamin C, retinyl palmitate, selenium, cysteine, methionine, citric acid, sodium sulfate and parabens. Examples of diluent include, but are not limited to, water, alcohol, saline solution, glycol, mineral oil and dimethyl sulfoxide (DMSO).

As used herein the term “donor” refers to a donor providing the exogenous cells or mitochondria. In some embodiments, the donor is not suffering from a disease or disorder or is not suffering from the same disease of disorder which the subject is afflicted. In certain embodiments, the donor is the subject and the cells and/or mitochondria are autologous. In other embodiments, the donor is not the subject, and the cells and mitochondria are allogeneic.

The term “exogenous” or “isolated exogenous” with regard to mitochondria refers to mitochondria that are from a source which is external to the recipient cell. For example, in some embodiments, exogenous mitochondria are derived or isolated from a donor cell which is different than the donor of the recipient cell. In some embodiments, the exogenous mitochondria are derived from or isolated from a donor cell from the same subject as the recipient cell. For example, exogenous mitochondria may be purified, isolated or obtained from a donor cell and thereafter introduced into a recipient cell from the same subject as the donor cell or a different donor, making the exogenous mitochondria autologous and allogeneic, respectively. In certain embodiments, the exogenous mitochondria are whole mitochondria.

In some embodiments, the exogenous donor cell is a stem cell and/or progenitor cell, a hematopoietic blast cell, or any combination thereof.

In some embodiments, the present invention provides a composition including stem cells derived from an MDS patient, enriched ex-vivo with isolated healthy mitochondria. The term “myelodysplastic syndrome” or “MDS” refers to a heterogeneous group of closely related clonal hematopoietic disorders commonly found in the aging population. It is known that during the aging process mitochondria accumulate mutations and deletions in the mitochondrial DNA becoming dysfunctional in multiple organ systems, including bone marrow. All are characterized by one or more peripheral blood cytopenia. Bone marrow is usually hypercellular, but rarely, a hypocellular marrow mimicking aplastic anemia may be seen. Bone marrow cells display aberrant morphology and maturation (dysmyelopoiesis), resulting in ineffective blood cell production. MDS affects hematopoiesis at the stem cell level, as indicated by cytogenetic abnormalities, molecular mutations, and morphologic and physiologic abnormalities in maturation and differentiation of one or more of the hematopoietic cell lines.

A myelodysplastic syndrome (MDS) is one of a group of diseases in which immature blood cells in the bone marrow do not mature, so do not become healthy blood cells. The term “MDS” encompasses multiple diseases and disorders, with some types capable to develop into acute myeloid leukemia.

The terms “disease” and “disorder” are meant to refer to any affliction that are not considered normal or that are different from a physiological state. Disease and disorders can affect virtually any organ, tissue, or function is the body. Non limiting examples of diseases and condition include cancer and blood diseases and disorders for example. As used herein the term “a subject afflicted with a disease or disorder” or “a subject having a disease or disorder” refers to a human subject experiencing debilitating effects caused by certain conditions. The disorder may refer to cancer, age related disorders, or blood disease, as well as other disease or disorders.

The term “MDS disease” or “MDS disorder” is to be understood as referring to any of the disease and disorder caused by blood cells that are poorly formed or don't function properly. Non-limiting example of MDS disease include the following:

Myelodysplastic syndrome with single-lineage dysplasia (MDS-SLD) occurs when one blood cell type, e.g., white blood cells, red blood cells or platelets, is low in number and appears abnormal under the microscope. This type of MDS is not common and rarely progresses to AML;

Myelodysplastic syndrome with multilineage dysplasia (MDS-MLD) occurs when two or three blood cell types are low in number and appear abnormal. This is the most common form of MDS;

Myelodysplastic syndrome with ring sideroblasts (MDS-RS) occurs when a low number of one or more blood cell types where red blood cells in the bone marrow contain rings of excess iron. MDS-RS is diagnosed when at least 15% of the early red blood cells are ring sideroblasts or at least 5% of the cells also have a mutation in the SF3B1 gene;

Myelodysplastic syndrome with isolated del(5q) chromosome abnormality occurs when there are low numbers of red blood cells, and the cells are missing part of chromosome 5. This type of MDS is not common and rarely progresses to AML;

Myelodysplastic syndrome with excess blasts (MDS-EB) occurs when any of the three types of blood cells might be low, appear abnormal under a microscope and very immature blood cells (blasts) are found in the blood and bone marrow. MDS-EB1 occurs when blast cells male up 5% to 9% of the cells in the bone marrow or 2% to 4% of the cells in the blood. MDS-EB2 occurs when blast cells make up 10% to 19% of the cells in the bone marrow or 5% to 19% of the cells in blood. This type of MDS is fairly common and is more likely to progress to AML;

Myelodysplastic syndrome, unclassifiable (MDS-U) occurs when there are reduced numbers of one or more types of mature blood cells and the cells might look abnormal under the microscope. Sometimes the blood cells appear normal, but analysis might find that the cells have DNA changes that are associated with myelodysplastic syndrome;

acute myeloid leukemia (AML); and

Chronic myelomonocytic leukemia (CMML).

As used herein, “leukemia” refers to a blood cancer caused by the rapid production of abnormal white blood cells. Examples of leukemia include acute lymphoblastic leukemia (ALL), acute myeloid leukemia (AML), chronic lymphocytic leukemia, chronic myelogenous leukemia, and hairy cell leukemia.

In one aspect, the stem cells and/or progenitor cells enriched with exogenous mitochondria are produced by a method including contacting the stem cells and/or progenitor cells with the exogenous mitochondria under conditions allowing the exogenous mitochondria to enter the stem cells and/or progenitor cells.

As used herein the term “contacting” refers to bringing the mitochondria and cells (for example, stem cells and/or progenitor cells) into sufficient proximity to promote entry of the exogenous mitochondria into the cells or stimulating the entry of exogenous mitochondria into the cells. The term introducing or inserting mitochondria into the cells (for example, stem cells and/or progenitor cells) is used interchangeably with the term contacting.

The phrase “conditions allowing the exogenous mitochondria to enter the stem cells” and “conditions allowing the exogenous mitochondria to enter the progenitor cells” as used herein generally refers to parameters such as time, temperature, centrifugation, culture medium and proximity between the mitochondria and the recipient cells. For example, human cells and human cell lines are routinely incubated in liquid medium, and kept in sterile environments, such as in tissue culture incubators, at 37° C. and 5% CO₂ atmosphere. According to alternative embodiments disclosed and exemplified herein the cells may be incubated at room temperature in saline supplemented with human serum albumin.

In some aspects, the conditions allowing the exogenous mitochondria to enter the stem cells and/or progenitor cells include incubating the stem cells and/or progenitor cells with the exogenous mitochondria for a time ranging from about 0.5 to 30 hours at a temperature ranging from about 4 to 37° C.

In certain embodiments, the cells are incubated with the exogenous mitochondria for a time ranging from 0.5 to 30 hours, at a temperature ranging from about 4 to 37° C. In certain embodiments, the cells are incubated with the exogenous mitochondria for a time ranging from about 0.5 to 30 hours or from about 5 to 25 hours. In specific embodiments, incubation is for about 0.5 to 20 hours. In specific embodiments, incubation is for about 20 to 30 hours. In some embodiments, incubation is for at least about 0.5, 1, 5, 10, 15, 20, 21, 22, 23 or 24 hours. In other embodiments, incubation is up to 5, 10, 15, 20 or 30 hours. In specific embodiments, incubation is for 24 hours. In certain embodiments, incubation is until the mitochondrial content in the cells is increased in average by 1% to 45% compared to their initial mitochondrial content.

In some embodiments, incubation is at room temperature (16° C. to 30° C.). In other embodiments, incubation is at 37° C. In certain embodiments, incubation is at 4° C. In some embodiments, incubation is in a 5% CO₂ atmosphere. In other embodiments, incubation does not include added CO₂ above the level found in air.

In yet further embodiments, the incubation is performed in culture medium. In some embodiments, the culture medium is supplemented with human serum albumin (HSA). In certain embodiments, the incubation is performed in a medium that maintains mitochondrial integrity. In additional embodiments, the incubation is performed in saline supplemented with HSA. According to certain exemplary embodiments, the conditions allowing the exogenous mitochondria to enter the human stem cells thereby enriching said human stem cells with said human exogenous mitochondria include incubation at room temperature in saline supplemented with 4.5% human serum albumin.

In certain embodiments, the incubation is performed at 37° C. In certain embodiments, the incubation is performed for at least 1 hour. In certain embodiments, the incubation is performed for at least 6 hours. In certain embodiments, the incubation is performed for at least 12 hours. In certain embodiments, the incubation is performed for 12 to 24 hours.

As used herein, the term “enriching” refers to any action designed to increase the mitochondrial content of a cell or a population of cells, e.g., the number of intact mitochondria, or the functionality of mitochondria of a mammalian cell or a population of cells. In some embodiments, stem cells and/or progenitor cells enriched with exogenous mitochondria will show enhanced function compared to the same stem cells and/or progenitor cells prior to enrichment.

The terms “enriching” and “enrichment” as used herein refer to any action performed ex vivo, which increases the mitochondrial content, e.g., the number of intact, functional, or healthy mitochondria, of a human cell or a population of cells. According to the principles of the present invention, exogenous mitochondria are introduced into human stem cells and/or progenitor cells, thus enriching these cells with exogenous mitochondria. According to some embodiments, the exogenous mitochondria constitute above 0.5%, 1%, 2%, 3%, 4%, 5%, 10%, 15% or 20% of the total mitochondria in the mitochondrially-enriched stem cells and/or progenitor cells.

As used herein the term “mitochondrial content” refers to the amount of mitochondria within a cell, or to the average amount of mitochondria within a plurality of cells. The term “increased mitochondrial content” as used herein refers to a mitochondrial content which is detectably higher than the mitochondrial content of the cells prior to mitochondria enrichment.

In certain embodiments, the mitochondrial content of the cells enriched with exogenous mitochondria is detectably higher than the mitochondrial content of the naïve cells. According to various embodiments, the mitochondrial content of the mitochondrially-enriched stem cells is at least 0.5%, 1%, 5%, 10%, 25%, 50%, 100%, 200% or more, higher than the mitochondrial content of the cells prior to mitochondria enrichment. In certain embodiments, the stem cells are used fresh. In certain embodiments, the stem cells and/or progenitor cells are frozen and thawed. In certain embodiments, the term “detectably higher” as used herein refers to a statistically-significant increase between the normal and increased values. In certain embodiments, the term “detectably higher” as used herein refers to a non-pathological increase, i.e., to a level in which no pathological symptom associated with the substantially higher value becomes apparent. In certain embodiments, the term “increased” as used herein refers to a value which is 1.05-fold, 1.1-fold, 1.25-fold, 1.5-fold, 2-fold, 3-fold, 4-fold, 5-fold, 6-fold, 7-fold or higher than the corresponding value found in corresponding cells prior to enrichment or corresponding mitochondria of a healthy subject or of a plurality of healthy subjects (for example, stem cells and progenitor cells) prior to mitochondrial enrichment.

In certain embodiments, the mitochondrial content of the cells or mitochondrially-enriched stem cells and/or progenitor cells is determined by determining the content of citrate synthase. In certain embodiments, the mitochondrial content of the naïve cells or enriched cells is determined by determining the activity level of citrate synthase. In certain embodiments, the mitochondrial content of the naive cells or enriched cells correlates with the content of citrate synthase. In certain embodiments, the mitochondrial content of the naive cells or enriched cells correlates with the activity level of citrate synthase. CS activity can be measured by commercially available kits.

In one aspect, the conditions allowing the exogenous mitochondria to enter the target cells include incubating the target cells with the exogenous mitochondria at a ratio of about 0.044-176 mU citrate synthase (CS) activity per 10⁶ cells.

Citrate synthase (CS) is localized in the mitochondrial matrix but is encoded by nuclear DNA. Citrate synthase is involved in the first step of the Krebs cycle and is commonly used as a quantitative enzyme marker for the presence of intact mitochondria (Larsen S. et al., J. Physiol., 2012, Vol. 590 (14), pages 3349-3360; Cook G. A. et al., Biochim. Biophys. Acta., 1983, Vol. 763 (4), pages 356-367).

Mitochondrial dose can be expressed in terms of units of CS activity or mtDNA copy number of other quantifiable measurements of the amount of mitochondria as explained herein. A “unit of CS activity” is defined as the amount that enables conversion of one micromole substrate in 1 minute in 1 mL reaction volume.

In some embodiments, the enrichment of the cells (for example, stem cells and/or progenitor cells) with exogenous mitochondria includes introducing into the cells a dose of mitochondria of at least 0.044 up to 176 milliunits (mU) of citrate synthase (CS) activity per million cells; at least 0.088 up to 176 mU of CS activity per million cells; at least 0.2 up to 150 mU of CS activity per million cells; at least 0.4 up to 100 mU of CS activity per million cells; at least 0.6 up to 80 mU of CS activity per million cells; at least 0.7 up to 50 mU of CS activity per million cells; at least 0.8 up to 20 mU of CS activity per million cells; at least 0.88 up to 17.6 mU of CS activity per million cells; or at least 0.44 up to 17.6 mU of CS activity per million cells.

In some aspects, the conditions allowing the exogenous mitochondria to enter the target cells include incubating the target cells with the exogenous mitochondria at a concentration of about 1-50 mU citrate synthase (CS) activity per 10⁶ cells. In various aspects, the concentration of exogenous mitochondria is about 0.88, 4.4, 17.6, or 35mU citrate synthase (CS) activity per 10⁶cells.

In some aspects, the concentration of exogenous mitochondria is at least 1 million up to 400 million mitochondria particles per million cells. In some embodiments, the concentration of exogenous mitochondria is at least 1 million up to 200 million mitochondria particles per million cells. In some embodiments, the concentration of exogenous mitochondria is at least 5 million up to 150 million mitochondria particles per million cells. In some embodiments, the concentration of exogenous mitochondria is at least 5 million up to 150 million mitochondria particles per million cells. In some embodiments, the concentration of exogenous mitochondria is at least 5 million up to 100 million mitochondria particles per million cells. In some embodiments, the concentration of exogenous mitochondria is at least 5 million up to 75 million mitochondria particles per million cells. In some embodiments, the concentration of exogenous mitochondria is at least 10 million up to 100 million mitochondria particles per million cells. In some embodiments, the concentration of exogenous mitochondria is at least 10 million up to 75 million mitochondria particles per million cells. In some embodiments, the concentration of exogenous mitochondria is at least 10 million up to 50 million mitochondria particles per million cells. In some aspects, the concentration of exogenous mitochondria is 1 million mitochondria particles per 1 million cells as measured by MTG method.

In another aspect, the stem cells and/or progenitor cells enriched with exogenous mitochondria have: an increased content of at least one mitochondrial protein; an increased rate of oxygen (O₂) consumption; an increased activity level of citrate synthase, succinate, or tryptamine; an increase rate of adenosine triphosphate (ATP) production; an increased mitochondrial DNA content; an increased colony forming unit activity in liquid medium or solid medium; an increased proliferation rate; an increased differentiation rate; or any combination thereof, as compared to the stem cells and/or progenitor cells prior to mitochondrial enrichment.

Mitochondrial DNA content may be measured by performing quantitative or digital PCR of a mitochondrial gene prior and post mitochondrial enrichment, normalized to a nuclear gene.

In specific situations the same cells, prior to mitochondria enrichment, serve as controls to determine enrichment level.

The term “increased mitochondrial DNA content” as used herein refers to the content of mitochondrial DNA which is detectably higher than the mitochondrial DNA content in cells prior to mitochondria enrichment. “Normal mitochondrial DNA” in the context of the specification and claims refers to mitochondrial DNA not carrying/having a mutation or deletion that is known to be associated with a mitochondrial disease. The term “normal rate of oxygen (O₂) consumption” as used herein refers to the average O₂ consumption of cells from healthy individuals. The term “normal activity level of citrate synthase” as used herein refers to the average activity level of citrate synthase in cells from healthy individuals. The term “normal rate of adenosine triphosphate (ATP) production” as used herein refers to the average ATP production rate in cells from healthy individuals.

According to some embodiments, the mitochondrial content is determined by measuring the content of a mitochondrial protein. According to some embodiments, a mitochondrial protein is a protein encoded by the mitochondrial DNA. According to some embodiments, a mitochondrial protein is a protein encoded by the nuclear DNA and localizes to the mitochondria. Non-limiting example are citrate synthase (CS), cytochrome C oxidase (COX1), succinate dehydrogenase complex flavoprotein subunit A (SDHA), tryptamine, succinate.

In some embodiments, the conditions allowing the exogenous mitochondria to enter the target cells comprise incubating the target cells with the exogenous mitochondria at a ratio of about 1 million up to 100 million mitochondria particles per 1 million cells. In certain embodiments, the conditions allowing the exogenous mitochondria to enter the target cells comprise incubating the target cells with the exogenous mitochondria at a ratio of about 10 million up to 50 million mitochondria particles per 1 million cells.

In some embodiments, the identification/discrimination of endogenous mitochondria from exogenous mitochondria, after the latter have been introduced into the target cell, can be performed by various means, including, for example, but not limited to identifying differences in mtDNA sequences, for example different haplotypes, between the endogenous mitochondria and exogenous mitochondria.

In certain embodiments, the endogenous and exogenous mitochondria are from identical haplogroups.

In other embodiments, the endogenous and exogenous mitochondria are from different haplogroups.

The extent of enrichment of the cells with exogenous mitochondria may be determined by functional and/or enzymatic assays, including but not limited to rate of oxygen (O₂) consumption, content or activity level of citrate synthase, rate of adenosine triphosphate (ATP) production. In some embodiments, the enrichment of the cells with exogenous mitochondria may be confirmed by the detection of mitochondrial DNA. According to some embodiments, the extent of enrichment of the cells with exogenous mitochondria may be determined by the level of change in heteroplasmy and/or by the copy number of mtDNA per cell.

Heteroplasmy is the presence of more than one type of mitochondrial DNA within a cell or individual. The heteroplasmy level is the proportion of mutant mtDNA molecules vs. wild type/functional mtDNA molecules and is an important factor in considering the severity of mitochondrial diseases. While lower levels of heteroplasmy (sufficient amount of mitochondria are functional) are associated with a healthy phenotype, higher levels of heteroplasmy (insufficient amount of mitochondria are functional) are associated with pathologies. In some embodiments, the heteroplasmy level of the mitochondrially-enriched stem cells is at least 1% lower than the heteroplasmy level of the stem cells prior to enrichment. In some embodiments, the heteroplasmy level of the mitochondrially-enriched stem cells is at least 3% lower than the heteroplasmy level of the stem cells prior to enrichment. In some embodiments, the heteroplasmy level of the mitochondrially-enriched stem cells is at least 5% lower than the heteroplasmy level of the stem cells prior to enrichment. In some embodiments, the heteroplasmy level of the mitochondrially-enriched stem cells is at least 10% lower than the heteroplasmy level of the stem cells prior to enrichment. In some embodiments, the heteroplasmy level of the mitochondrially-enriched stem cells is at least 20% lower than the heteroplasmy level of the stem cells prior to enrichment. In some embodiments, the heteroplasmy level of the mitochondrially-enriched stem cells is at least 30% lower than the heteroplasmy level of the stem cells prior to enrichment. In some embodiments, the heteroplasmy level of the mitochondrially-enriched stem cells is at least 50% lower than the heteroplasmy level of the stem cells prior to enrichment.

TMRM (tetramethyl rhodamine methyl ester) or the related TMRE (tetramethyl rhodamine ethyl ester) are cell-permeant fluorogenic dyes commonly used to assess mitochondrial function in living cells, by identifying changes in mitochondrial membrane potential. According to some embodiments, the level of enrichment can be determined by staining with TMRE or TMRM. Other fluorogenic dyes well known in the art can also be used.

According to some embodiments, the mitochondria include intact mitochondria, ruptured mitochondria and/or mitochondrial constituents selected from the group consisting of mitochondrial protein, mitochondrial nucleic acid, mitochondrial lipid and mitochondrial saccharide.

According to some embodiments, the intactness of a mitochondrial membrane may be determined by any method known in the art. In a non-limiting example, intactness of a mitochondrial membrane is measured using the cytochrome c release test, tetramethyl rhodamine methyl ester (TMRM), or the tetramethyl rhodamine ethyl ester (TMRE) fluorescent probes. Each possibility represents a separate embodiment of the present invention. Mitochondria that were observed under a microscope and show TMRM or TMRE staining have an intact mitochondrial outer membrane. As used herein, the term “a mitochondrial membrane” refers to a mitochondrial membrane selected from the group consisting of the mitochondrial inner membrane, the mitochondrial outer membrane, and both.

In certain embodiments, the level of mitochondrial enrichment in the mitochondrially-enriched stem cells and/or progenitor cells is determined by sequencing at least a statistically representative portion of total mitochondrial DNA in the cells and determining the relative levels of host/endogenous mitochondrial DNA and exogenous mitochondrial DNA. In certain embodiments, the level of mitochondrial enrichment in the mitochondrially-enriched stem cells and/or progenitor cells is determined by single nucleotide polymorphism (SNP) analysis. In certain embodiments, the largest mitochondrial population and/or the largest mitochondrial DNA population is the host/endogenous mitochondrial population and/or the host/endogenous mitochondrial DNA population; and/or the second-largest mitochondrial population and/or the second-largest mitochondrial DNA population is the exogenous mitochondrial population and/or the exogenous mitochondrial DNA population.

According to certain embodiments, the enrichment of the stem cells and/or progenitor cells with exogenous mitochondria may be determined by conventional assays that are recognized in the art. In certain embodiments, the level of mitochondrial enrichment in the mitochondrially-enriched human stem cells and/or progenitor cells is determined by (i) the levels of host/endogenous mitochondrial DNA and exogenous mitochondrial DNA; (ii) the level of a mitochondrial protein (iii) the level of CS activity; or (iv) any combination of (i), (ii) and (iii). Methods for determining these various parameters are well known in the art.

In certain embodiments, the level of mitochondrial enrichment in the mitochondrially-enriched stem cells and/or progenitor cells is determined by at least one of: (i) the levels of host mitochondrial DNA and exogenous mitochondrial DNA; (ii) the level of citrate synthase activity; (iii) the level of succinate dehydrogenase complex flavoprotein subunit A (SDHA) or cytochrome C oxidase (COX1); (iv) the rate of oxygen (O₂) consumption; (v) the rate of adenosine triphosphate (ATP) production or (vi) any combination thereof. Each possibility represents a separate embodiment of the present invention. Methods for measuring these various parameters are well known in the art.

In some embodiments, enrichment of the cells with exogenous human mitochondria includes washing the mitochondrially-enriched cells (for example mitochondrially-enriched stem cells and/or progenitor cells) after incubation of the cells with the exogenous human mitochondria. This step provides mitochondrially-enriched cells substantially devoid of cell debris or mitochondrial membrane remnants and mitochondria that did not enter the stem cells and/or progenitor cells. In some embodiments, washing includes centrifugation of the mitochondrially-enriched cells after incubation of the human cells with said exogenous human mitochondria. According to some embodiments, the methods produce mitochondrially-enriched cells that are separated from free mitochondria, i.e., mitochondria that did not enter the cells, or other cell debris and the pharmaceutical compositions contain mitochondrially-enriched cells that are separated from free mitochondria. According to some embodiments, the methods produce, and the pharmaceutical compositions contain mitochondrially-enriched stem cells and/or progenitor cells that do not comprise a detectable amount of free mitochondria.

The term “increased rate of oxygen (O₂) consumption” as used herein refers to a rate of oxygen (O₂) consumption which is detectably higher than the rate of oxygen (O₂) consumption prior to mitochondria enrichment.

The term “increased content of at least one mitochondrial protein” as used herein refers to the content of either nuclear-encoded or mitochondrial-encoded mitochondrial proteins, e.g., CS, COX1 and SDHA, which is detectably higher than the content of said mitochondrial protein in the cells prior to mitochondrial enrichment.

The term “increased content or activity level of citrate synthase” as used herein refers to a content or activity level of citrate synthase which is detectably higher than the content value or activity level of citrate synthase in cells prior to mitochondrial enrichment.

The term “increased rate of adenosine triphosphate (ATP) production” as used herein refers to a rate of adenosine triphosphate (ATP) production which is detectably higher than the rate of adenosine triphosphate (ATP) production prior to mitochondria enrichment.

In one aspect, the exogenous mitochondria constitute at least 0.5% of the total mitochondria in the stem cells and/or progenitor cells enriched with exogenous mitochondria.

In certain embodiments, the exogenous mitochondria constitute at least 0.5% of the total mitochondria content in the mitochondrially-enriched cells. In certain embodiments, the exogenous mitochondria constitute at least 10% of the total mitochondria content in the mitochondrially-enriched target cells. In some embodiments, the exogenous mitochondria constitute at least about 0.5%, 1%, 2%, 3%, 5%, 10%, 15%, 20%, 25%, 30%, 40% or 50% of the total mitochondria content in the mitochondrially-enriched target cells. In certain embodiments, the total amount of mitochondrial proteins in the isolated mitochondria, is between 10-90%, 20-80%, 20-70%, 40-70%, 20-40%, or 20-30% of the total amount of cellular proteins. Each possibility represents a separate embodiment of the present invention. In certain embodiments, the total amount of mitochondrial proteins in the isolated mitochondria, is between 20%-80% of the total amount of cellular proteins within the sample. In certain embodiments, the total amount of mitochondrial proteins in the isolated mitochondria, is between 10%-80% of the combined weight of the mitochondria and other sub-cellular fractions. In other embodiments, the total amount of mitochondrial proteins in the isolated mitochondria, is above 80% of the combined weight of the mitochondria and other sub-cellular fractions.

In another aspect, the exogenous mitochondria are isolated, derived or partially purified human mitochondria.

As used herein, the terms “isolated”, “derived” and “partially purified” in the context of mitochondria includes exogenous mitochondria that were purified, at least partially, from other cellular components. The total amount of mitochondrial proteins in an exogenous isolated or partially purified mitochondria is between about 10%-90% of the total amount of cellular proteins within the sample.

As used herein the term “functional mitochondria” refers to mitochondria displaying parameters indicative of normal mitochondrial DNA (mtDNA) and/or normal, non-pathological levels of activity. The activity of mitochondria can be measured by a variety of methods well known in the art, such as membrane potential, O₂ consumption, ATP production, and citrate synthase (CS) activity level.

In one aspect, the composition is frozen/thawed. In some aspects, the stem cells and/or progenitor cells are frozen/thawed prior to the enrichment. In other aspects, the exogenous mitochondria are frozen/thawed prior to the enrichment of the stem cells and/or progenitor cells. In another aspect, the stem cells and/or progenitor cells have undergone at least one freeze-thaw cycle after enrichment with the exogenous mitochondria.

In some embodiments, the stem cells and/or progenitor cells are cultured and expanded in vitro. In certain embodiments, the stem cells and/or progenitor cells undergo at least one freeze thaw cycle prior to or following mitochondrial enrichment.

In certain embodiments, the stem cells and/or progenitor cells are frozen then stored and used after thawing. In further embodiments, the exogenous mitochondria are frozen, then stored and thawed prior to use. In further embodiments the mitochondrially-enriched stem cells and/or progenitor cells are used without freezing and storage. In yet further embodiments, the mitochondrially-enriched stem cells and/or progenitor cells are used after freezing, storage and thawing. Methods suitable for freezing and thawing of cell preparations in order to preserve viability are well known in the art.

As used herein, the term “freeze-thaw cycle” refers to freezing of the exogenous mitochondria to a temperature below 0° C., maintaining the mitochondria in a temperature below 0°° C. for a defined period of time and thawing the exogenous mitochondria to room temperature or body temperature or any temperature above 0° C. which enables treatment of the cells with the exogenous mitochondria. The term “room temperature”, as used herein typically refers to a temperature of between 18° C. and 25° C. The term “body temperature”, as used herein, refers to a temperature of between 35.5° C. and 37.5° C., preferably 37° C.

In another embodiment, the mitochondria that have undergone a freeze-thaw cycle were frozen at a temperature of −20° C. or lower; −4° C. or lower; or −70° C. or lower. According to another embodiment, freezing of the mitochondria is gradual. According to some embodiment, freezing of mitochondria is through flash freezing. As used herein, the term “flash-freezing” refers to rapidly freezing the mitochondria by subjecting them to cryogenic temperatures.

In another embodiment, the mitochondria that underwent a freeze-thaw cycle were frozen for at least 30 minutes prior to thawing. According to another embodiment, the freeze-thaw cycle includes freezing the exogenous mitochondria for at least 30, 60, 90, 120, 180, 210 minutes prior to thawing. Each possibility represents a separate embodiment of the present invention. In another embodiment, the mitochondria that have undergone a freeze-thaw cycle were frozen for at least 1, 2, 3, 4, 5, 6, 7, 8, 9, 10, 24, 48, 72, 96, or 120 hours prior to thawing. In another embodiment, the mitochondria that have undergone a freeze-thaw cycle were frozen for at least 4, 5, 6, 7, 30, 60, 120, 365 days prior to thawing. According to another embodiment, the freeze-thaw cycle includes freezing the exogenous mitochondria for at least 1, 2, 3 weeks prior to thawing. According to another embodiment, the freeze-thaw cycle includes freezing the exogenous mitochondria for at least 1, 2, 3, 4, 5, 6, 12 months prior to thawing. Each possibility represents a separate embodiment of the present invention.

According to certain embodiment, thawing is at room temperature. In another embodiment, thawing is at body temperature. According to another embodiment, thawing is at a temperature which enables administering the mitochondria according to the methods of the invention. According to another embodiment, thawing is performed gradually.

In another aspect, the stem cells and/or progenitor cells are selected from the group consisting of pluripotent stem cells, embryonic stem cells, induced pluripotent stem cells, hematopoietic stem cells, hematopoietic progenitor cells, common myeloid progenitor cells, common lymphoid progenitor cells, CD34 ⁺ cells, a subset of CD34 ⁺ cells, and any combination thereof.

In certain embodiments, the stem cells are pluripotent stem cells (PSC). In other embodiments, the PSCs are non-embryonic stem cells. In some embodiments, the stem cells are induced PSCs (iPSCs). In certain embodiments, the stem cells are embryonic stem cells. In certain embodiments, the stem cells are derived from bone-marrow cells. In particular embodiments, the stem cells are CD34 ⁺ cells. In yet other embodiments, the stem cells are derived from blood. In further embodiments, the stem cells are derived from umbilical cord blood. In specific embodiments, the stem cells obtained from a subject afflicted with a disease of disorder or from a healthy subject are bone marrow cells or bone marrow-derived stem cells.

As used herein the term “pluripotent stem cells (PSCs)” refers to cells that can propagate indefinitely, as well as give rise to a plurality of cell types in the body. Totipotent stem cells are cells that can give rise to every other cell type in the body. Embryonic stem cells (ESCs) are totipotent stem cells and induced pluripotent stem cells (iPSCs) are pluripotent stem cells.

As used herein the term “induced pluripotent stem cells (iPSCs)” refers to a type of pluripotent stem cell that can be generated from human adult somatic cells. Some non-limiting examples of somatic cells from which iPSC can be generated herein include hematopoietic stem cells or progenitor cells thereof.

As used herein the term “embryonic stem cells (ESC)” refers to a type of totipotent stem cell derived from the inner cell mass of a blastocyst.

As used herein the term “bone marrow cells” generally refers to all human cells naturally found in the bone marrow of humans, and to all cell populations naturally found in the bone marrow of humans. The term “bone marrow stem cells” and “bone marrow-derived stem cells” refer to the stem cell and/or progenitor stem cell population derived from the bone marrow.

In some embodiments, the autologous or allogeneic human stem cells are pluripotent stem cells (PSCs) or induced pluripotent stem cells (iPSCs).

According to several embodiments, the human stem cells are derived from blood, umbilical cord blood, bone marrow or bone marrow cells mobilized to blood. Each possibility represents a separate embodiment of the present invention. In specific embodiments, the human stem cells are derived from bone marrow.

In certain embodiments, the bone-marrow derived stem cells include myelopoietic cells. The term “myelopoietic cells” as used herein refers to cells involved in myelopoiesis, e.g., in the production of bone-marrow and of all cells that arise from it, namely, all blood cells.

In certain embodiments, the bone-marrow derived stem cells include erythropoietic cells. The term “erythropoietic cells” as used herein refers to cells involved in erythropoiesis, e.g., in the production of red blood cells (erythrocytes).

In certain embodiments, the bone-marrow derived stem cells include multi-potential hematopoietic stem cells (HSCs). The term “multi-potential hematopoietic stem cells” or “hemocytoblasts” as used herein refers to the stem cells that give rise to all the other blood cells through the process of hematopoiesis.

In certain embodiments, the bone-marrow derived stem cells include common myeloid progenitor cells, common lymphoid progenitor cells, or any combination thereof. In certain embodiments, the bone-marrow derived stem cells include mesenchymal stem cells. The term “common myeloid progenitor” as used herein refers to the cells that generate myeloid cells. The term “common lymphoid progenitor” as used herein refers to the cells that generate lymphocytes.

In certain embodiments, the bone-marrow derived stem cells further include megakaryocytes, erythrocytes, mast cells, myoblasts, basophils, neutrophils, eosinophils, monocytes, macrophages, natural killer (NK) cells, small lymphocytes, T lymphocytes, B lymphocytes, plasma cells, reticular cells, or any combination thereof. Each possibility represents a separate embodiment of the invention.

In certain embodiments, the bone-marrow derived stem cells include myelopoietic cells. In certain embodiments, the bone-marrow derived stem cells consist of erythropoietic cells. In certain embodiments, the bone-marrow derived stem cells include multi-potential hematopoietic stem cells (HSCs). In certain embodiments, the bone-marrow derived stem cells include common myeloid progenitor cells, common lymphoid progenitor cells, or any combination thereof. In certain embodiments, the bone-marrow derived stem cells include megakaryocytes, erythrocytes, mast cells, myoblasts, basophils, neutrophils, eosinophils, monocytes, macrophages, natural killer (NK) cells, small lymphocytes, T lymphocytes, B lymphocytes, plasma cells, reticular cells, or any combination thereof. In certain embodiments, the stem cells include a plurality of human bone marrow stem cells obtained from peripheral blood.

Hematopoietic stem cells (HSCs) are the stem cells that give rise to other blood cells. This process is called hematopoiesis. Hematopoietic stem cells give rise to different types of blood cells, in lines called myeloid and lymphoid. Myeloid and lymphoid lineages both are involved in dendritic cell formation. Myeloid cells include monocytes, macrophages, neutrophils, basophils, eosinophils, erythrocytes, and megakaryocytes to platelets. Lymphoid cells include T cells, B cells, and natural killer cells.

In one aspect, the stem cells and/or progenitor cells are derived from whole blood, blood fractions, peripheral blood, umbilical blood, bone marrow, or bone marrow cells mobilized to blood.

In various aspects, the stem cells and/or progenitor cells are CD34 ⁺ cells.

Hematopoietic progenitor cell antigen CD34, also known as CD34 antigen, is a protein that in humans is encoded by the CD34 gene. CD34 is a cluster of differentiation in a cell surface glycoprotein and functions as a cell-cell adhesion factor. In certain embodiments, the stem cells express the bone-marrow progenitor cell antigen CD34 (are CD34 ⁺). In certain embodiments, the stem cells do not express CD34 (are CD34). In certain embodiments, the stem cells present the bone-marrow progenitor cell antigen CD34 on their external membrane. In certain embodiments the CD34 ⁺ cells are from umbilical cord blood. As used herein the term “CD34 ⁺ cells” refers to hematopoietic stem cells characterized as being CD34 positive, regardless of their origin. In certain embodiments, the CD34 ⁺ cells are obtained from the bone marrow, from bone marrow cells mobilized to the blood, or obtained from umbilical cord blood.

CD34 is used to identify and isolate human hematopoietic stem cells and progenitor cells, for example for use in bone marrow transplantation. The compositions described herein include CD34 ⁺ stem cells and progenitor cells, enriched with exogenous mitochondria. Once engrafted, the mitochondria-enriched CD34 ⁺ stem cells and progenitor cells can expand and form new colonies, and repopulate the subject bone marrow with healthy hematopoietic cells, that differentiate from the stem cells and progenitor cells.

In certain embodiments, the stem cells, including hematopoietic stem cells, are obtained from the peripheral blood of the subject afflicted with a disease or disorder. In certain embodiments, the stem cells are obtained from the peripheral blood of a healthy donor. The term “peripheral blood” as used herein refers to blood circulating in the blood system.

As used herein, the term “autologous cells” or “cells that are autologous”, refers to being the subject's own cells. The term “autologous mitochondria” refers to mitochondria obtained from the subject's own cells or from maternally related cells. The terms “allogeneic cells” or “allogeneic mitochondria”, refer to cells or mitochondria being from a different donor individual.

The stem cells used in the present compositions are CD34 ⁺ cells or subset of CD34 ⁺ cells obtained from a healthy donor or a patient with any stage of MDS disease.

In some aspects, the MDS disease or disorder is selected from the group consisting of myelodysplastic syndrome with single-lineage dysplasia (MDS-SLD), myelodysplastic syndrome with multilineage dysplasia (MDS-MLD), myelodysplastic syndrome with ring sideroblasts (MDS-RS), myelodysplastic syndrome with isolated del(5q), myelodysplastic syndrome with excess blasts (MDS-EB), myelodysplastic syndrome, unclassifiable (MDS-U), and acute myeloid leukemia (AML).

Subject with myelodysplastic syndrome might not experience signs and symptoms at first. In time, myelodysplastic syndrome might cause various non-specific symptoms. In one aspect, the MDS symptom is selected from the group consisting of shortness of breath, weakness, fatigue, paleness, anemia, thrombocytopenia, leukopenia, bruising, bleeding, petechiae, ineffective hematopoiesis, blood cytopenia, clonal instability, and any combination thereof.

The compositions described herein includes stem cells obtained from a subject having MDS, regardless of the treatment history of the subject. The stem cells can be from a patient that has been pretreated for MDS and that does not undergo treatment anymore, from a patient that is currently treated for the symptoms of the MDS, or from a patient that has never been treated yet.

In one aspect, the subject is treated or has been treated with a MDS treatment.

For subject currently undergoing treatment, the present composition can be administered in combination with one or more MDS treatment. The phrases “combination therapy”, “combined with” and the like refer to the use of more than one medication or treatment simultaneously to increase the response. The composition of the present invention might for example be used in combination with other drugs or treatment in use to treat cancer. Specifically, the administration of the composition of the present invention to a subject can be in combination with any anti-MDS therapies. Such therapies can be administered prior to, simultaneously with, or following administration of the composition of the present invention.

Treatment in low-risk MDS (LR-MDS) focuses mainly on improving cytopenia and quality of life (QoL). Early intervention with current approaches has not shown mortality benefit nor impact on reducing clonal evolution in LR-MDS. The goal of treatment in high-risk MDS (HR-MDS) is to prevent disease progression and improve survival. For eligible patients, allogeneic hematopoietic cell transplantation (HCT) is the only potentially curative treatment. The standard of care treatment for HR-MDS patients who are not candidates for HCT is DNA hypomethylating agents (HMA) until disease progression or intolerance. Available MDS treatment are well-known in the art (see for example myelodysplastic syndrome current treatment algorithm 2018 at www.nature.com/articles/s41408-018-0085-4.pdf) and include the orally administered immunomodulatory drug lenalidomide; nucleoside analogs that are DNA hypomethylating agents (azacitidine, decitabine and cedazuridine/decitabine); erythroid maturation agent luspatercept, and the oral hypomethylating agent cedazuridine/decitabine. In addition to these agents, there is extensive off-label use of the erythropoiesis stimulating agents (ESA) epoetin and darbepoetin in MDS. Hematopoietic cell transplantation (HCT) is the only curative option, but a majority of patients are not eligible due to age and comorbidities. Other alternative or supplemental treatment include a Combination of Coenzyme Q10 and Carnitine, the enhancement of mitochondrial function and ESA-injections IV/SC (such as lenalidomide and luspatercept).

In some aspects, the MDS treatment is a hypomethylating agent, an erythropoiesis stimulating agent (ESA), granulocyte colony-stimulating factor (G-CSF), azacytidine, decitabine, an immunosuppressive therapy (IST), luspatercept, or a combination thereof.

In another aspect, the exogenous mitochondria are isolated, derived or purified from human cells. In some embodiments, the exogenous mitochondria are isolated, derived or purified from placenta, placental cells grown in culture, or blood cells.

In certain embodiments, the exogenous mitochondria are obtained from a human cell or a human tissue. In certain embodiments, the cells are selected from the group consisting of placenta, placental cells grown in culture, or blood cells. In some embodiments, the mitochondria are obtained from human stem cells. In some embodiments, the human cell is a human somatic cell. In some embodiments, the human cell are cells grown in culture.

In some aspects, the present invention provides stem cells derived from an MDS patient, enriched ex-vivo with isolated healthy mitochondria, and methods of using the enriched cells for treating MDS diseases, disorders and symptoms thereof.

In one embodiment, the invention provides a method of reducing a myelodysplastic syndrome (MDS) disease or disorder-associated symptom in a subject including administering to the subject stem cells and/or progenitor cells enriched with exogenous mitochondria.

In one aspect, the subject has one or more MDS disease-or disorder-associated symptoms.

In various aspects, the one or more MDS disease-or disorder-associated symptoms are selected from the group consisting of shortness of breath, weakness, fatigue, paleness, anemia, thrombocytopenia, leukopenia, bruising, bleeding, petechiae, ineffective hematopoiesis, blood cytopenia, bone marrow dysplasia, lymphopenia clonal instability, and any combination thereof.

In an additional embodiment, the invention provides a method of preventing myelodysplastic syndrome (MDS) disease or disorder progression in a subject including administering to the subject stem cells and/or progenitor cells enriched with exogenous mitochondria.

In a further embodiment, the invention provides a method of treating a myelodysplastic syndrome (MDS) disease or disorder in a subject including administering to the subject stem cells and/or progenitor cells enriched with exogenous mitochondria.

The term “treatment” is used interchangeably herein with the term “therapeutic method” and refers to both 1) therapeutic treatments or measures that cure, slow down, lessen symptoms of, and/or halt progression of a diagnosed pathologic conditions or disorder, and 2) and prophylactic/preventative measures. Those in need of treatment may include individuals already having a particular medical disorder as well as those who may ultimately acquire the disorder (i.e., those needing preventive measures).

The terms “therapeutically effective amount”, “effective dose,” “therapeutically effective dose”, “effective amount,” or the like refer to that amount of the subject compound that will elicit the biological or medical response of a tissue, system or human that is being sought by the researcher, medical doctor or other clinician. Generally, the response is either amelioration of symptoms in a patient or a desired biological outcome (e.g., reducing a myelodysplastic syndrome (MDS) disease or disorder-associated symptom). Such amount should be sufficient to reduce a myelodysplastic syndrome (MDS) disease or disorder-associated symptom. The effective amount can be determined as described herein.

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intravenous, intraperitoneal, intraarterial, intramuscular, infusion and direct injection to bone marrow. The phrases “parenteral administration” and “administered parenterally” as used herein means modes of administration other than enteral and topical administration. The pharmaceutical compositions can be administered in a variety of unit dosage forms depending upon the method of administration. Suitable unit dosage forms include, but are not limited to capsules, injectables, implantable sustained-release formulations, and lipid complexes. In another aspect, administration of mitochondrially-enriched stem cells and/or progenitor cells is by intravenous, intraperitoneal, intraarterial or intramuscular administration.

In one aspect, administering stem cells and/or progenitor cells enriched with exogenous mitochondria prevents disease progression and/or improves survival.

By “preventing disease progression” and “improving survival”, it is meant that the mitochondria-enriched stem cells allow the stabilization or the amelioration of the disease or its symptoms, such that a subject that has a reduced or limited survival because of the disease increases such survival. For example, the mitochondria-enriched stem cells allow the restoration of the levels of red blood cells (RBC), platelets (PLT), and white blood cells (WBC), the recovery of normal or normalized blood counts and mononuclear cell differentiation. Such restoration and recovery are part of the restoration of a functional hematopoietic lineage. In certain embodiments, restoring hematopoietic function or cell count is characterized by an improved cell differentiation, an improvement of anemia, reduction in the number of blast cells, a reduction in the number of ring sideroblasts and/or a reduction for the need for a blood transfusion.

In one aspect, the improved cell differentiation includes improved CD34 ⁺ cells erythroid differentiation.

In one embodiment, the invention provides a method of restoring hematopoietic lineage function in a subject in need thereof including administering to the subject stem cells and/or progenitor cells enriched with exogenous mitochondria.

In one aspect, the stem cells and/or progenitor cells are autologous.

The term “stem cells are autologous to the subject” as used herein refers to being the subject's own cells. The cells are isolated from a subject and undergo ex vivo modification (e.g., enrichment in healthy mitochondria). These mitochondria-enriched stem cells are then expanded, selected if necessary, and infused into the subject. After transplantation, the mitochondria-enriched stem cells undergo amplification in the peripheral blood.

In another aspect, the stem cells and/or progenitor cells enriched with exogenous mitochondria colonize the bone marrow to establish new hematopoietic colonies.

In another aspect, the method further includes administering to the subject a MDS treatment selected from the group consisting of a hypomethylating agent, an erythropoiesis stimulating agent (ESA), an erythroid maturation agent, an immunosuppressive therapy (IST), a growth factor, an immunomodulatory drug, a nucleoside analog, a blood transfusion, a bone marrow transplant, or a combination thereof. In some aspects, the MDS treatment is selected from the group consisting of azacytidine, decitabine, cedazuridine, luspatercept, granulocyte colony-stimulating factor (G-CSF), lenalidomide, epoetin and darbepoetin, or a combination thereof.

In one aspect, the exogenous mitochondria constitute at least 1% of the total mitochondria content in the mitochondrially-enriched stem cells and/or progenitor cells.

In one aspect, between at least 5×10⁵ to 5×10⁹ mitochondrially-enriched stem cells and/or progenitor cells are administered to the subject.

Presented below are examples discussing mitochondrial enrichment in stem and progenitor cells contemplated for the discussed applications. The following examples are provided to further illustrate the embodiments of the present invention but are not intended to limit the scope of the invention. While they are typical of those that might be used, other procedures, methodologies, or techniques known to those skilled in the art may alternatively be used

### EXAMPLES

### Example 1

### In Vitro Effect of Mitochondrial Enrichment in MDS Hematopoietic Stem/Progenitor Cells

MDS is a disorder in which HSPC differentiation is blocked before terminal differentiation. Ineffective hematopoiesis in MDS results from increased susceptibility of clonal myeloid progenitors to apoptosis. This may be triggered by intrinsic factors such as mitochondrial polarization due to iron retention in ringed sideroblasts. Patient cells have mtDNA mutations and lower O₂ consumption rates. Preclinical mouse polg model (which amasses mitochondrial mutations/deletions) demonstrates that mitochondrial dysfunction of HSC is causal to MDS phenotypes and is cell intrinsic.

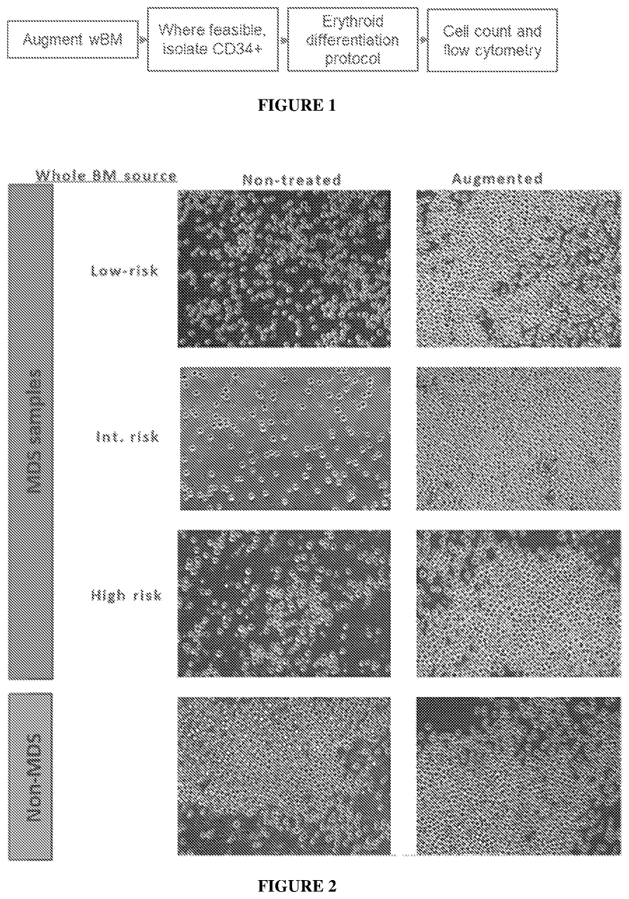

To assess the effect of mitochondrial enrichment in vitro, the proliferation, differentiation potential and survival of MDS hematopoietic stem/progenitor cells in vitro was measured, along with the evaluation of the mitochondrial function of the enriched cells. Preclinical evidence suggested that MAT promotes HSPC differentiation and proliferation to B cell lineage. It was thus assessed whether MAT allowed differentiation of HSPCs from MDS patients to erythroid and/or platelet lineages to alleviate cytopenias, following the study design illustrated in FIG. 1. CD34 ⁺ cells were collected from blood and then augmented with mitochondria.

CD34 ⁺ cells were isolated from bone marrow mononuclear cells (BM MNCs) of MDS patients having early or late stage MDS.

Enrichment process:

The CD34 ⁺ cells (1M cells per mL) were treated with or without mitochondria isolated from placenta or from another source. The mitochondria were at a concentration of 0.88, 4.4, 17.6,35 mU CS activity per 1M cells.

TABLE 1
Example of samples assessment

| | | | Female/ | MDS | mU CS activity per |
|---|---|---|---|---|---|
| | Sample # | Patient | Male | Stage | 1M cells |
| Treated | 1, 2, 3, 4 | 1 | F | Early | 0.88, 4.4, 17.6, 35 |
| Untreated | 5 | 1 | F | Early | — |
| Control | 6 | 1 | F | Early | |
| Treated | 7, 8, 9, 10 | 2 | F | Early | 0.88, 4.4, 17.6, 35 |
| Untreated | 11 | 2 | F | Early | — |
| Control | 12 | 2 | F | Early | |
| Treated | 13, 14, 15, 16 | 3 | M | Early | 0.88, 4.4, 17.6, 35 |
| Untreated | 17 | 3 | M | Early | — |
| Control | 18 | 3 | M | Early | |
| Treated | 19, 20, 21, 22 | 4 | M | Early | 0.88, 4.4, 17.6, 35 |
| Untreated | 23 | 4 | M | Early | — |
| Control | 24 | 4 | M | Early | |
| Treated | 25, 26, 27, 28 | 5 | F | Late | 0.88, 4.4, 17.6, 35 |
| Untreated | 29 | 5 | F | Late | — |
| Control | 30 | 5 | F | Late | |
| Treated | 31, 32, 33, 34 | 6 | F | Late | 0.88, 4.4, 17.6, 35 |
| Untreated | 35 | 6 | F | Late | — |
| Control | 36 | 6 | F | Late | |
| Treated | 37, 38, 39, 40 | 7 | M | Late | 0.88, 4.4, 17.6, 35 |
| Untreated | 41 | 7 | M | Late | — |
| Control | 42 | 7 | M | Late | |
| Treated | 43, 44, 45, 46 | 8 | M | Late | 0.88, 4.4, 17.6, 35 |
| Untreated | 47 | 8 | M | Late | — |
| Control | 48 | 8 | M | Late | |

The CD34 cells were incubated in vitro in liquid culture medium (StemSpan SFEM II media) comprising Erythroid Expansion Supplement (StemSpan™ Erythroid Expansion Supplement) to induce Erythroid lineage or Megakaryocyte Supplement (StemSpan Megakaryocyte Expansion Supplement) to induce Megakaryocytes+ platelets lineage. The medium was replaced every 3 days.

Flow cytometric analysis was performed every 3 days in liquid culture for ˜14 days of culture. Flow cytometric analysis was performed to enumerate hematopoietic stem and progenitor cells using CD34 marker. To enumerate myeloid precursor cells the Megakaryocytes markers of CD41a platelets and CD42b were used. To enumerate erythroid precursor cells GlycophorinA (APC) and CD71 (erythroid precursor) markers were used.

Additionally, to assess CFU formation the CD34 cells are incubated in vitro in semi-solid agar culture medium (MethoCult Media for CFU Assays) to induce differentiation to CFU-GM, CFU-GEMM, BFU-E, CFU-E colonies. Colonies were identified and quantified after 14+/−2 days.

Bone marrow cells from MDS were augmented with 4.4 mU CS Hela-GFP mitochondria (FIG. 2, right panel) or not treated (FIG. 2, left panel) (non-treated cells are cells which underwent the process of augmentation without the addition of mitochondria). Augmentation percentage was evaluated using flow analysis. Low risk and intermediate risk MDS derived cells (top two rows) underwent CD34 ⁺ purification post augmentation following which exposed to erythroid lineage differentiation supplement. NT and Augmented groups were cultured at 20,000 total cells each for 14 days. High risk MDS patient cells did no undergo CD34 ⁺ purification due to low number of cells and directly exposed to erythroid differentiation supplement for 14 days (100,000 cells each). Medium was replenished every 3 days and cells were counted using nucleocounter at the end of the culturing and verified for CD71/CD235 surface marker expression.

As illustrated in FIG. 2, MAT positively affected the erythroid differentiation capacity of MDS-HSC derived bone marrow samples.

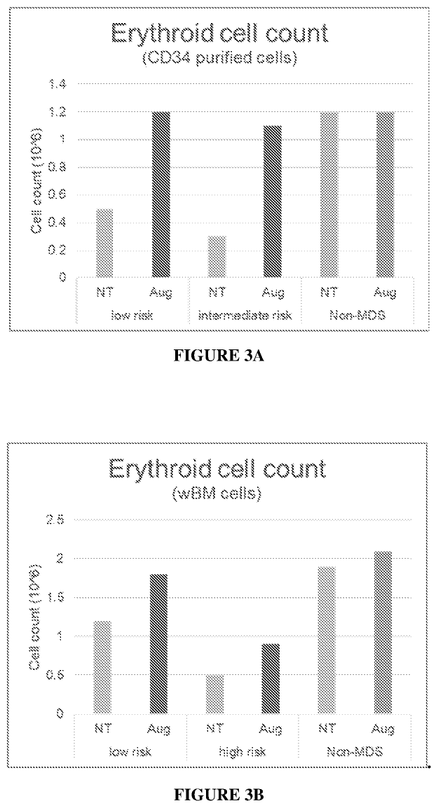

As illustrated in FIGS. 3A and 3B and in Tables 2 and 3, MAT improved MDS-CD34 ⁺ erythroid differentiation, as shown with the higher levels of differentiation to erythroid lineage observed after augmentation of MDS patient-derived cells with GFP-mito (n=3).

TABLE 2

| | | | Erythroid cells (Million | |
|---|---|---|---|---|
| Sample (isolated | | | cells) | |
| CD34) | % CD34+ | % GFP+ | NT | Aug |
| Low risk | 5.3 | 2 | 0.5 | 1.2 |
| Intermediate risk | 15.4 | 7.5 | 0.3 | 1.1 |
| Non-MDS | 5.8 | 3.4 | 1.2 | 1.2 |

TABLE 3

| | | | Erythroid cells (Million | |
|---|---|---|---|---|
| Sample (isolated | | | cells) | |
| CD34) | % CD34+ | % GFP+ | NT | Aug |
| Low risk | 5.3 | 2 | 0.5 | 1.2 |
| Intermediate risk | 15.4 | 7.5 | 0.3 | 1.1 |
| Non-MDS | 5.8 | 3.4 | 1.2 | 1.2 |

### Example 2

### In Vivo Effect of Mitochondrial Enrichment in MDS Hematopoietic Stem/Progenitor Cells

To assess the impact of mitochondrial enrichment in vivo genetically engineered models of MDS are used.

Genetically engineered mouse models of MDS including mice with conditional deletion of Tet2 and/or Asxl1, as well as conditional expression of MDS-associated mutations in Sf3b1(SF3B1K700E and K666N mutations), Srsf2 (SRSF2P95H), or Zrsr2 (Zrsr2 floxed mice). In particular, the Sf3b1 and Srsf2 mutant mice have highly penetrant lymphopenia, macrocytosis, and impaired hematopoietic stem cells (HSC) reconstitution ability which mimic key aspects of human MDS. In the Sf3b1 model there is a mutation in a spliceosome gene; a gene that is associated with most human MDS with BM ringed sideroblasts. However, the mouse model doesn't recapitulate ringed sideroblasts. It is associated with progressive anemia.

Additional mouse model to be used includes the NUP98-HOXD13 (also known as NHD13). NHD13 faithfully comprises all of the key features of MDS, including peripheral blood cytopenia, bone marrow dysplasia, and apoptosis, and transformation to acute leukemia; it inhibits megakaryocytic differentiation and increases apoptosis in the bone marrow. The MDS that develops in NUP98-HOXD13 transgenic mice is uniformly fatal within 14 months.

Another mouse model includes NSG humanized mouse with augmented patient-derived MDS cells.

Mitochondria are isolated from placenta or liver of wild-type (WT) control mice of a strain selected from Mx1-cre, C57, WSP, W8, CAST or NZB. The mitochondria are frozen in Liquid Nitrogen or at −80° C. HSPCs are isolated from 8-week-old CD45.2+ Mx1-cre Sf3b1 mutant or Srsf2^{P95/WT}mutant mice or NHD13 mice. The mitochondria are thawed and then the cells are incubated with or without 0.88, 4.4, 17.6, or 35 mU CS activity per 1M cells for up to 24 hours. Subsequently, the media is removed, and cells are washed and resuspended in 4.5% albumin, in a physiological cell suspension media. Using sequence analysis, augmentation is verified by identifying the presence of the exogenous mitochondria in the cells using sequencing methods.

Competitive bone marrow transplantation assays are performed in CD45.1 recipient mice using CD45.1/CD45.2 double-positive competitor cells mixed with equal numbers of cells from CD45.2+ Mx1-cre Sf3b1mutant, Srsf2^{P95/WT} mutant or NDH13 mutant mice, with or without mitochondrial enrichment. Recipient mice are bled monthly for 16 weeks post-transplantation to evaluate the impact of mitochondria enrichment on CD45.2 chimerism in peripheral blood and differentiation of CD45.2 cells. Specifically, time to restoration and final levels of red blood cells (RBC), platelets (PLT), and white blood cells (WBC) are monitored.

In addition, the impact of mitochondrial enrichment on hematopoietic stem and progenitor subsets in the bone marrow at 16 weeks post-transplant is evaluated by assessing the levels of lin⁻ cells and Lin⁻SCA1+c-KIT⁺ (LSK) cells. Persistence of exogenous mitochondria is also monitored.

To measure the impact of MAT on long-term self-renewal, serial competitive transplantations are conducted by isolating LSK or lin cells from animals treated with enriched or non-enriched lin cells and transplanting to a naïve animal as described above.

In addition to the above competitive transplantation assays, noncompetitive transplantations of BM MNCs are also performed, with and without mitochondrial enrichment from 8-week-old CD45.2+ Mx1-cre Sf3b1 mutant or Srsf2^{P95/WT} mutant or NHD13 mutant mice into lethally irradiated CD45.1 wild-type recipient mice. Recipient mice are then bled monthly to evaluate the impact of mitochondria enrichment on blood counts and MNC differentiation. In these assays, Mx1-cre Sf3b1 mutant or Srsf2^{P95/WT} mutant or NHD13 mutant cells have lymphopenia and macrocytic anemia. The impact of mitochondria enrichment on these parameters is therefore evaluated in particular.

### Example 3

### Effect OF Mitochondrial Enrichment in Hematopoietic Stem/Progenitor Cells for the Treatment of MDS

To assess the impact of mitochondrial enrichment in patients with MDS, stem cells and/or progenitor cells are obtained from mobilized peripheral blood or directly from the bone marrow of subjects having a myelodysplastic syndrome (MDS) disease, disorder or symptom. Exogenous mitochondria are obtained from a donor that does not have a MDS disease, disorder or symptom thereof. The stem cells and/or progenitor cells are contacted with the exogenous mitochondria under conditions allowing the exogenous mitochondria to enter some or all of the stem cells and/or progenitor cells thereby producing a composition comprising mitochondria-enriched stem cells and/or progenitor cells.

The subjects are administered with the mitochondria-enriched stem cells and/or progenitor cells. The prevention of disease progression, improvement of survival and reduction of symptoms are evaluated in subjects administered with the mitochondria-enriched stem cells and/or progenitor cells, as compared to control subjects (e.g., subjects not administered with mitochondria-enriched stem cells and/or progenitor cells) by measuring variation in MDS disease or disorder-associated symptoms. For example, hematopoiesis, blood cytopenia, clonal instability, lymphopenia, macrocytosis, hematopoietic stem cells (HSC) reconstitution, peripheral blood cytopenia, bone marrow dysplasia, macrocytic anemia, apoptosis, transfusion necessity frequency, and transformation to acute leukemia are monitored, as well as blood counts and MNC differentiation (e.g., restoration and final levels of red blood cells (RBC), platelets (PLT), and white blood cells (WBC)). The subjects may be administered subsequent doses of mitochondria-enriched stem cells and/or progenitor cells. The subjects may also receive chemotherapy prior to being administered mitochondria-enriched stem cells and/or progenitor cells.

Although the invention has been described with reference to the above examples, it will be understood that modifications and variations are encompassed within the spirit and scope of the invention. Accordingly, the invention is limited only by the following claims.

1. A pharmaceutical composition comprising stem cells and/or progenitor cells enriched with exogenous mitochondria and a pharmaceutically acceptable carrier,
wherein the stem cells and/or progenitor cells are obtained from a subject having a myelodysplastic syndrome (MDS) disease, disorder or symptom thereof, and
wherein the exogenous mitochondria are obtained from a donor that does not have a MDS disease, disorder or symptom thereof or a mitochondrial disease.

2. The pharmaceutical composition of claim 1, wherein the stem cells and/or progenitor cells enriched with exogenous mitochondria are produced by contacting the stem cells and/or progenitor cells with the exogenous mitochondria under conditions allowing the exogenous mitochondria to enter the stem cells and/or progenitor cells.

3. The pharmaceutical composition of claim 1, wherein the conditions allowing the exogenous mitochondria to enter the stem cells and/or progenitor cells comprise incubating the stem cells and/or progenitor cells with the exogenous mitochondria for a time ranging from about 0.5 to 30 hours at a temperature ranging from about 4 to 37° C.

4. The pharmaceutical composition of claim 1, wherein the conditions allowing the exogenous mitochondria to enter the target cells comprise incubating the target cells with the exogenous mitochondria at a ratio of about 0.044-176 mU citrate synthase (CS) activity per 10⁶ cells.

5-6. (canceled)

7. The pharmaceutical composition of claim 1, wherein the conditions allowing the exogenous mitochondria to enter the target cells comprise incubating the target cells with the exogenous mitochondria at a ratio of about 1million up to 100 million mitochondria particles per 1 million cells.

8. (canceled)

9. The pharmaceutical composition of claim 1, wherein the stem cells and/or progenitor cells enriched with exogenous mitochondria have:
a) an increased content of at least one mitochondrial protein;
b) an increased rate of oxygen (O₂) consumption;
c) an increased activity level of citrate synthase, succinate, or tryptamine;
d) an increase rate of adenosine triphosphate (ATP) production;
e) an increased mitochondrial DNA content;
f) an increased colony forming unit activity in liquid medium or solid medium;
g) an increased proliferation rate;
h) an increased differentiation rate; and
i) any combination thereof,
as compared to the stem cells and/or progenitor cells prior to mitochondrial enrichment.

10. The pharmaceutical composition of claim 1, wherein the exogenous mitochondria constitute at least 0.5% of the total mitochondria in the stem cells and/or progenitor cells enriched with exogenous mitochondria.

11. The pharmaceutical composition of claim 1, wherein the exogenous mitochondria are isolated, derived or partially purified human mitochondria.

12-14. (canceled)

15. The pharmaceutical composition of claim 1, wherein the stem cells and/or progenitor cells have undergone at least one freeze-thaw cycle after enrichment with the exogenous mitochondria.

16. The pharmaceutical composition of claim 1, wherein the stem cells and/or progenitor cells are selected from the group consisting of pluripotent stem cells, embryonic stem cells, induced pluripotent stem cells, hematopoietic stem cells, hematopoietic progenitor cells, common myeloid progenitor cells, common lymphoid progenitor cells, CD34 ⁺ cells, a subset of CD34 ⁺ cells and any combination thereof.

17. The pharmaceutical composition of claim 1, wherein the stem cells and/or progenitor cells are CD34 ⁺ cells or a subset of CD34 ⁺ cells.

18. The pharmaceutical composition of claim 1, wherein the stem cells and/or progenitor cells are derived from whole blood, blood fractions, peripheral blood, umbilical blood, bone marrow, or bone marrow cells mobilized to blood.

19. The pharmaceutical composition of claim 1, wherein the exogenous mitochondria are isolated, derived or purified from placenta, placental cells grown in culture or blood cells.

20. The pharmaceutical composition of claim 1, wherein the MDS disease or disorder is selected from the group consisting of myelodysplastic syndrome with single-lineage dysplasia (MDS-SLD), myelodysplastic syndrome with multilineage dysplasia (MDS-MLD), myelodysplastic syndrome with ring sideroblasts (MDS-RS), myelodysplastic syndrome with isolated del(5q), myelodysplastic syndrome with excess blasts (MDS-EB), myelodysplastic syndrome, unclassifiable (MDS-U), and acute myeloid leukemia (AML).

21. The pharmaceutical composition of claim 1, wherein the MDS symptom is selected from the group consisting of shortness of breath, weakness, fatigue, paleness, anemia, thrombocytopenia, leukopenia, bruising, bleeding, petechiae, ineffective hematopoiesis, blood cytopenia, clonal instability, and any combination thereof.

22. (canceled)

23. The pharmaceutical composition of claim 22, wherein the MDS treatment is a hypomethylating agent, an erythropoiesis stimulating agent (ESA), granulocyte colony-stimulating factor (G-CSF), azacytidine, decitabine, an immunosuppressive therapy (IST), luspatercept, or a combination thereof.

24-29. (canceled)

30. A method of restoring hematopoietic lineage function or cell count in a subject in need thereof comprising administering to the subject stem cells and/or progenitor cells enriched with exogenous mitochondria, thereby restoring hematopoietic lineage function in the subject.

31. The method of claim 30, wherein restoring hematopoietic function or cell count is characterized by an improved cell differentiation, an improvement of anemia, reduction in the number of blast cells, a reduction in the number of ring sideroblasts and/or a reduction for the need for a blood transfusion.

32. The method of claim 31, wherein the improved cell differentiation comprises improved CD34 ⁺ cells erythroid differentiation.

33-35. (canceled)

36. The method of claim 30, wherein the stem cells and/or progenitor cells enriched with exogenous mitochondria colonize the bone marrow to establish new hematopoietic colonies.

37. The method of claim 30, further comprising administering to the subject a MDS treatment selected from the group consisting of a hypomethylating agent, an erythropoiesis stimulating agent (ESA), an erythroid maturation agent, an immunosuppressive therapy (IST), a growth factor, an immunomodulatory drug, a nucleoside analog, a blood transfusion, a bone marrow transplant, or a combination thereof.

38-41. (canceled)
